(19) United States
(12) Patent Application Publication
(10) Pub. No.: US 2009/0163945 A1
(43) Pub. Date: Jun. 25, 2009

(54) POLYMERIC SLOTTED TUBE COILS

(75) Inventors: Robert E. Richard, Wrentham, MA (US); Todd H. Turnlund, Park City, UT (US)

Correspondence Address: David T. Burse;Vista IP Law Group LLP, Suite D-2, 12930 Saratoga Avenue, Saratoga, CA, 95070, US

(73) Assignee: Boston Scientific Scimed, Inc., Maple Grove, MA (US)

(21) Appl. No.: 12/339,858

(22) Filed: Dec. 19, 2008

Related U.S. Application Data
(60) Provisional application No. 61/015,649, filed on Dec. 20, 2007.

(51) Int. Cl. A61M 29/04; A61M 29/00

(52) U.S. Cl. 606/191

(57) ABSTRACT

A vaso-occlusive device comprises a flexible tubular structure configured by creating slots on a tubular structure. The slots are configured such that connecting elements are produced between resulting adjacent segments of the slotted tubular structure. The connecting elements are preferably parallel to the opening within the vaso-occlusive tubular structure. The slotted tubular structure design provides flexibility as well as specific tie points for attachment of thrombogenic fibers.

### RELATED APPLICATION DATA

The present application claims the benefit under 35 U.S.C. §119 to U.S. provisional patent application Ser. No. 61/015,649, filed Dec. 20, 2007. The foregoing application is hereby incorporated by reference into the present application in its entirety.

### FIELD OF INVENTION

The field of the invention pertains to implantable devices, and more particularly to vaso-occlusive devices for the occlusion of body lumens and cavities such as aneurysms.

### BACKGROUND

In many clinical situations, blood vessels are occluded for a variety of purposes, such as to control bleeding, to prevent blood supply to tumors, or to block blood flow within an aneurysm.

Vaso-occlusive coils have been used in the treatment of aneurysms, and are generally constructed by winding a wire strand about a first, relatively small diameter mandrel (and then heat treating) to give it a helical coil “primary” (or delivery) shape, and then winding the helical coil around a much larger mandrel having various protrusions (and then heat treating) in order to produce a secondary shape that the coil takes once it is released at the vascular site. It is well known to attach thrombogenic fibers to the coils, which requires that the coils are stretched in order to allow attachment of the fibers. However, the stretching affects the shape memory of the coils.

### SUMMARY

Embodiments of the present invention are directed to vaso-occlusive devices that can be deployed within the vasculature to occlude the flow of blood therein. In particular, a flexible tubular structure is configured by creating slots on a tubular structure. The slots are sized and configured such that connecting elements are produced between resulting adjacent segments of the slotted tubular structure. The connecting elements are preferably parallel to the inner lumen of tubular structure. The slotted tubular structure design provides flexibility as well as specific tie points for attachment of thrombogenic fibers, without affecting any shape memory characteristics.

In one embodiment, the vaso-occlusive slotted tubular structure comprises biostable or biodegradable materials. In another embodiment, the vaso-occlusive slotted tubular structure is impregnated or coated with therapeutics to accelerate embolization or for other benefits. In another embodiment, the vaso-occlusive slotted tubular structure is water swellable to expand into the space where it is deployed. In another embodiment, the vaso-occlusive slotted tubular structure is lubricious to enhance deliverability. In another embodiment, the vaso-occlusive slotted tubular structure contains chemical sites capable of chelating radioisotopes for readioembolization applications.

### BRIEF DESCRIPTION OF THE DRAWINGS

The drawings illustrate the design and utility of embodiments of the present invention, in which similar elements are referred to by common reference numerals. In order to better appreciate the advantages and objects of the embodiments of the present invention, reference should be made to the accompanying drawings that illustrate these embodiments. However, the drawings depict only some embodiments of the invention, and should not be taken as limiting its scope. With this caveat, embodiments of the invention will be described and explained with additional specificity and detail through the use of the accompanying drawings, in which:

FIGS. 1-3 illustrate the creation of a first pair of opposing slots on a portion of a vaso-occlusive tubular structure, in accordance with one embodiment.

FIGS. 4-5 illustrate the creation of a second pair of opposing slots on the vaso-occlusive tubular structure adjacent to the first pair of opposing slots.

FIG. 6 illustrates a vaso-occlusive tubular structure configured with a plurality of slots and connecting elements, constructed in accordance with one embodiment of the present invention.

FIG. 7 illustrates the application of one or more filamentary elements to the slots and/or connecting elements on the vaso-occlusive tubular structure in FIG. 6.

FIG. 8 illustrates a delivery catheter configured for delivering a vaso-occlusive device constructed according to embodiments of the invention into the vasculature of a patient.

### DETAILED DESCRIPTION

Reference in this specification to “one embodiment” or “an embodiment” means that a particular feature, structure, or characteristic described in connection with the embodiment is included in at least one embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments. In general, features described in one embodiment might be suitable for use in other embodiments as would be apparent to those skilled in the art.

The present embodiments are directed to vaso-occlusive devices deployed to provide emboli in aneurysms located within the vasculatures of humans. The vaso-occlusive devices may also be used at any site in a human or animal that requires occlusion. The vaso-occlusive devices can be carried to a target site using a catheter and released therefrom using any one of a variety of detachable means, such as an electrolytic joint.

A flexible tubular structure is configured by configuring slots on a tubular structure such that connecting elements are produced between resulting adjacent segments of the slotted tubular structure. The slotted tubular structure design provides flexibility as well as specific tie points for attachment of thrombogenic fibers.

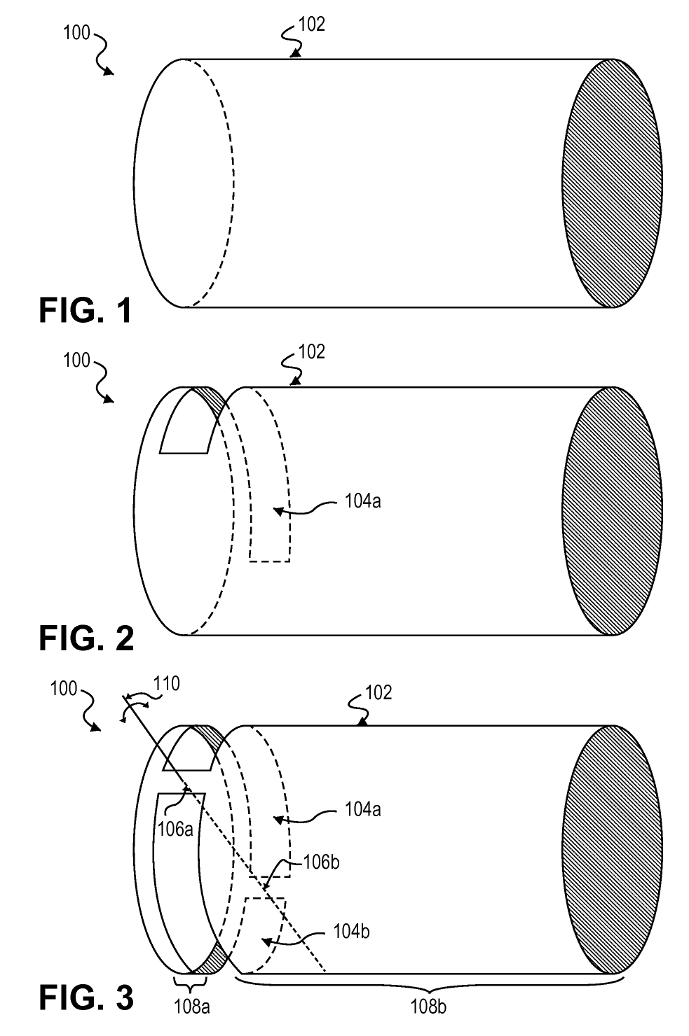

Referring to FIGS. 1-8, various embodiments of vaso-occlusive devices 100 will be described. The devices 100 comprise a flexible elongate vaso-occlusive tubular structure 102 and a plurality of slots 104 formed within the tubular structure 102. As will be described in further detail below, the configuration of the slots 104 allows thrombogenic fibers to be attached to the vaso-occlusive tubular structure 102. Additionally, the slots 104 weaken the axial and flexural strength of the tubular structure 102 and cause the vaso-occlusive tubular structure 102 so be more susceptible to bending or folding when the device 100 is subjected to an external force, for example when the device 100 comes in contact with an object, such as the wall of a body cavity.

In one embodiment, the vaso-occlusive tubular structure 102 has a circular cross-sectional shape. Alternatively, the vaso-occlusive tubular structure 102 may have rectangular, triangular, or other geometric cross-sections, or an irregularly shaped cross-section.

In one embodiment, the vaso-occlusive tubular structure 102 is made of a polymeric tube. The polymer tube can be made from biostable or biodegradable materials and can be impregnated or coated with therapeutics to accelerate embolization or for other benefits. The polymer can also be water swellable to expand into the space where it is deployed. The polymer can also be lubricious to enhance deliverability. The polymer can also, in addition to structural thrombogenic properties, contain chemical sites capable of chelating radioisotopes for radioembolization applications. The chelating can be a coating or surface treatment of the tube material. The polymer can be a shape memory polymer to allow secondary shapes to be annealed into it.

Biodegradable or absorbable materials suitable for use in the compositions of the vaso-occlusive tubular structure 102 include, but are not limited to, polymers and proteins. Suitable polymers include, for example, polyglygolic acid (PGA), poly-glycolic/poly-L-lactic acid co-polymers, polycaprolactone, polyhydroxybutyrate/hydroxyvalerate copolymers, poly-L-lactide, polydioxanone, polycarbonates, and polyanhydrides. Non-limiting examples of bioabsorbable proteins include collagen, elastin, fibrinogen, fibronectin, vitronectin, laminin and gelatin. Many of these materials are commercially available. Fibrin-containing compositions are commercially available, for example from Baxter. Collagen containing compositions are commercially available, for example, from Cohesion Technologies, Inc., Palo Alto, Calif. Fibrinogen-containing compositions are described, for example, in U.S. Pat. Nos. 6,168,788 and 5,290,552. As will be readily apparent, absorbable materials can be used alone or in any combination with each other. Absorbable materials used for the fibers attached to the coil (but not the coil itself) may be in the form of a mono-filament or, alternatively, a multi-filament strands.

Furthermore, the absorbable materials may be used in combination with additional components. For example, lubricious (e.g. hydrophilic) materials may be used to coat the tubular structure 102 to help facilitate delivery. One or more bioactive materials may also be included in the composition of the vaso-occlusive tubular structure 102. The term “bioactive” refers to any agent that exhibits effects in vivo, for example a thrombotic agent, a therapeutic agent or the like. Non-limiting examples of bioactive materials include cytokines; extracellular matrix molecules (e.g., collagen); trace metals (e.g., copper); and other molecules that stabilize thrombus formation or inhibit clot lysis (e.g., proteins or functional fragments of proteins, including but not limited to Factor XIII, α₂-antiplasmin, plasminogen activator inhibitor-1 (PAI-1) or the like). Non-limiting examples of cytokines which may be used alone or in combination in the practice of the present invention include, basic fibroblast growth factor (bFGF), platelet derived growth factor (pDGF), vascular endothelial growth factor (VEGF), transforming growth factor beta (TGF-β) and the like. Cytokines, extracellular matrix molecules, and thrombus stabilizing molecules are commercially available from several vendors such as, for example, Genzyme (Framingham, Mass.), Genentech (South San Francisco, Calif.), Amgen (Thousand Oaks, Calif.), R&D Systems and Immunex (Seattle, Wash.). Additionally, bioactive polypeptides can be synthesized recombinantly. The sequence of many of these molecules are available, for example, from the GenBank database. Thus, it is intended that the invention include use of DNA or RNA encoding any of the bioactive molecules. Furthermore, it is intended that molecules having similar biological activity as wild-type or purified cytokines, extracellular matrix molecules and thrombus-stabilizing proteins (e.g., recombinantly produced or mutants thereof) and nucleic acid encoding these molecules may also be used. The amount and concentration of the bioactive materials that may be included in the composition of the vaso-occlusive tubular structure 102 may vary, depending on the specific application, and can be readily determined by one skilled in the art. It will be understood that any combination of materials, concentration or dosage can be used, so long as it is not harmful to the subject.

For the compositions of the vaso-occlusive tubular structure 102, it may also be desirable to include one or more radiopaque materials for use in visualizing the vaso-occlusive tubular structure 102 in situ. Thus, the vaso-occlusive tubular structure 102 may be coated or mixed with radiopaque materials such as metals (e.g. tantalum, gold, tungsten or platinum), barium sulfate, bismuth oxide, bismuth subcarbonate, and the like.

Alternatively, the vaso-occlusive tubular structure 102 may be made of non-biodegradable materials, such as metals or alloys, for examples, that are in general more elastic than the biodegradable materials described previously. Suitable metals and alloys for the metal making up the tube include the Platinum Group metals, especially platinum, rhodium, palladium, rhenium, as well as tungsten, gold, silver, tantalum, and alloys of these metals. These metals have significant radiopacity and their alloys may be tailored to accomplish an appropriate blend of flexibility and stiffness. They are also largely biologically inert. Additional coating materials, such as polymer, or biodegradable materials as discussed previously, may be added to the surface of the vaso-occlusive tubular structure 102 to improve the thrombogenic properties of the vaso-occlusive device 100.

The vaso-occlusive tubular structure 102 may also be of any of a wide variety of stainless steels if some sacrifice of radiopacity may be tolerated. Very desirable materials of construction, from a mechanical point of view, are materials which maintain their shape despite being subjected to high stress. Certain “super-elastic alloys” include nickel/titanium alloys, copper/zinc alloys, or nickel/aluminum alloys, which are well-known in the art.

A titanium/nickel alloy known as “nitinol” may also be used in the vaso-occlusive tubular structure 102. These are super-elastic and very sturdy alloys that will tolerate significant flexing without deformation even when used as a very small diameter tube. If nitinol is used in the device, the diameter of the vaso-occlusive tubular structure 102 may be significantly smaller than that of a vaso-occlusive tubular structure 102 that uses the relatively more ductile platinum or platinum/tungsten alloy as the material of construction.

As illustrated in FIGS. 1-7, slots 104 are configured on the vaso-occlusive tubular structure 102. There are a number of methods for creating the slots 104 along the vaso-occlusive tubular structure 102. For example, the slots 104 may be created by laser-cutting or by mechanical or chemical removal of a portion of the vaso-occlusive tubular structure 102. The slots 104 may also be created during the fabrication of the vaso-occlusive tubular structure 104. Heat may also be used to shape the slots 104 in the vaso-occlusive tubular structure 102.

The sequence of FIGS. 1-3 illustrates the creation of one pair of opposing slots 104a and 104b on a portion of the vaso-occlusive tubular structure 102. Referring specifically to FIG. 1, a section of the vaso-occlusive tubular structure 102 is shown prior to the creation of any slots. FIG. 2 shows the section of the vaso-occlusive tubular structure 102 with a single slot 104a created along a portion of the structure 102. FIG. 3 shows the section of the vaso-occlusive tubular structure 102 with a second slot 104b created opposite the first slot 104a. The pair of opposing slots 104a and 104b creates two connecting elements 106a and 106b between the two resulting adjacent segments 108a and 108b of the tubular structure 102 that are on either side of the two opposing slots 104a and 104b. As a result, the two adjacent segments 108a and 108b can move relative to each other about the axis of rotation 110.

As will be presently described in more detail, a plurality of pairs of opposing slots 104 are created along the length of the vaso-occlusive tubular structure 102, with adjacent pairs of opposing slots 104 rotated relative to each other such that the resulting series of connecting elements 106 are also rotated relative to their adjacent neighbors, thereby allowing the vaso-occlusive tubular structure to bend in a plurality of directions.

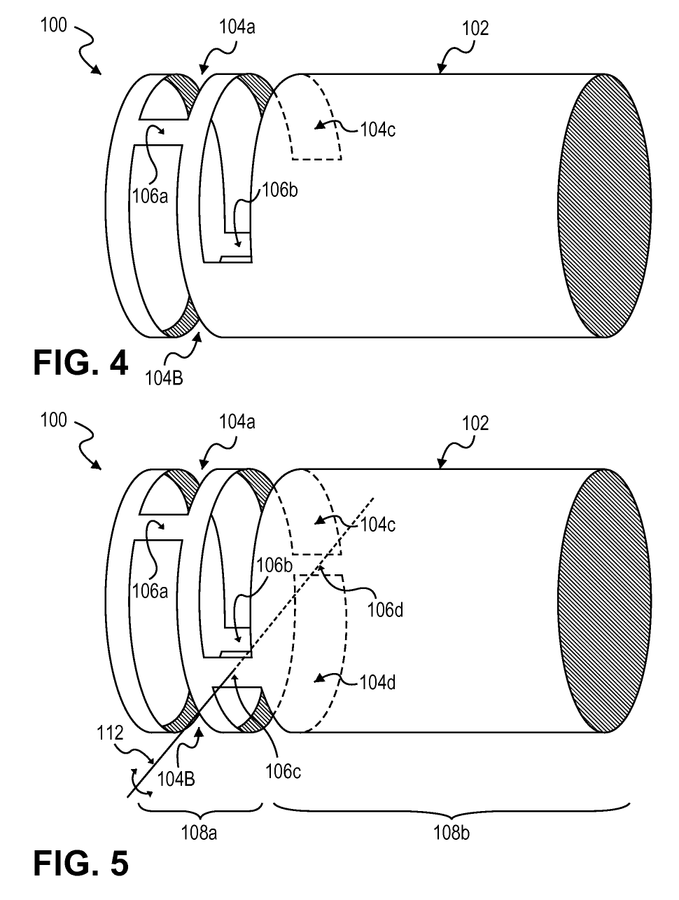

The sequence of FIGS. 4-5 illustrates the creation of a second pair of opposing slots 104c and 104d on the vaso-occlusive tubular structure 102 adjacent to the first pair of opposing slots 104a and 104b. Referring specifically to FIG. 4, a slot 104c is created adjacent to and rotated relative to the first pair of opposing slots 104a and 104b. Referring to FIG. 5, a second slot 104d is created opposite slot 104c, with the pair creating two connecting elements 106c and 106d rotated relative to the first pair of connecting elements 106a and 106b. As a result, the two resulting adjacent segments 108c and 108d of the vaso-occlusive tubular structure 102 can move relative to each other about the axis or rotation 112.

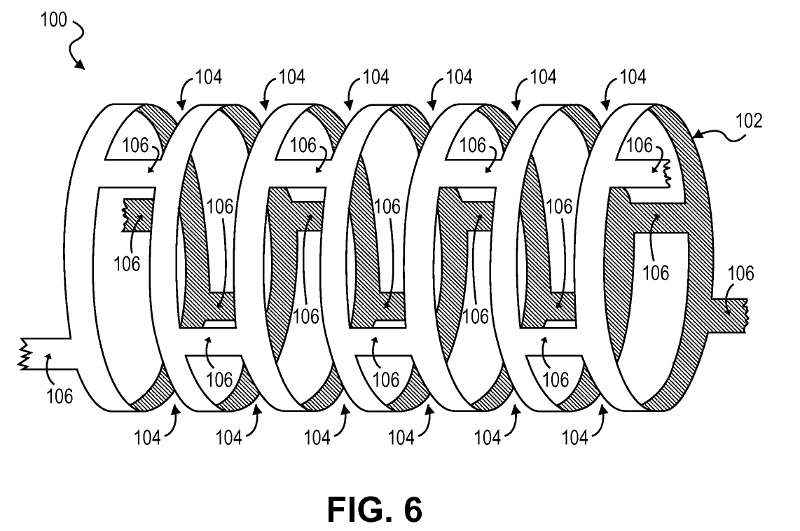

FIG. 6 illustrates a vaso-occlusive tubular structure 102 configured with a plurality of slots 104 and connecting elements 106. Advantageously, the configuration of the slots 104 and the connecting elements 106 allows the structure 102 to behave flexibly. The structure 102 may fold or bend to assume secondary shapes. In effect, this converts a primary shape into a secondary shape simply from the configuration of the slots 104 and connecting pieces 106, without the need for winding or otherwise manipulating the tubular structure 102 into a secondary shape. This is in contrast to conventional thrombogenic coils that are wound to assume a secondary shape.

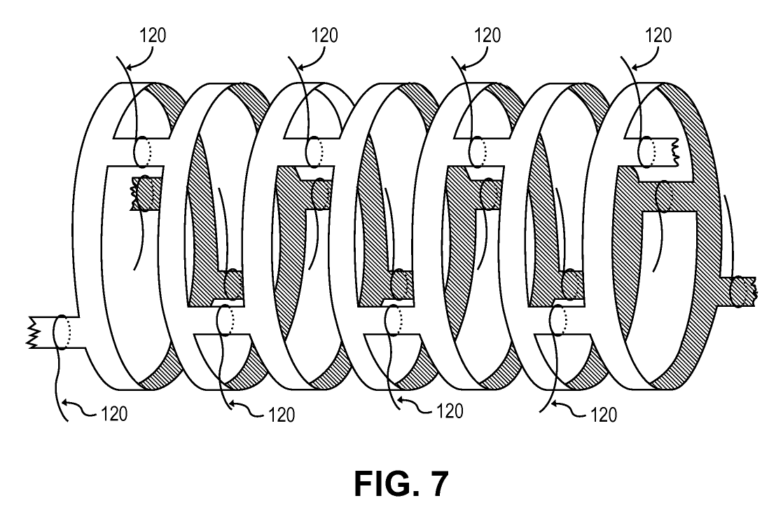

As shown in FIG. 7, the configuration of the slots 104 and connecting elements 106 on the vaso-occlusive tubular structure 102 permits easy application of one or more filamentary elements 120. The application of filamentary elements 120 increases the overall thrombogenic properties of the vaso-occlusive device 100. While conventional thrombogenic coils also permit attachment of fibers, such attachment is manual, time-consuming, and involves stretching the coils and tying the fibers to the stretched coils, which stretching affects the shape memory of the coils. In contrast, the present vaso-occlusive device 100 allows attachment of filamentary elements 120 to the connecting elements 106, as well as to the segments of the vaso-occlusive tubular structure 102 connected by the connecting elements 106, without having to stretch the vaso-occlusive tubular structure 102.

Fiber placement can be precisely and regularly controlled on the vaso-occlusive device 100 by attaching fibers to the connecting elements 106. Furthermore, the shape of the vaso-occlusive tubular structure 102 given by the slots 104 and connecting elements 106 inhibits movement of any attached fibers along the length of the vaso-occlusive tubular structure 102 due to sliding. Using the connecting elements 106 as the attachment points of the fibers 120 allows the optional use of automated fiber attaching processes such as knitting or sewing machines which greatly reduce the time to apply fibers to a vaso-occlusive device. In another optional embodiment, fibers 120 are weaved loosely through the slotted tubular structure 102, for example by using a weaving machine.

While the present vaso-occlusive tubular structure 102 can bend, it does not stretch. Hence, in contrast to conventional thrombogenic coils that stretch in a high-flow environment such as in a vessel with fast blood-flow, an advantageous aspect of the present vaso-occlusive device 100 is that the tubular structure 102 does not stretch in such a high-flow environment.

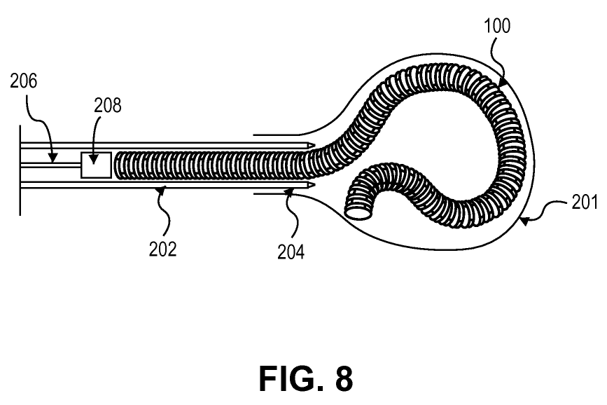

A method for using the previously described vaso-occlusive device 100 will now be discussed with reference to FIG. 8. First, a delivery catheter 202 is inserted into the body of a patient. Typically, this would be through a femoral artery in the groin. Other entry sites sometimes chosen are found in the neck and are in general well known by physicians who practice such medical procedures.

The delivery catheter 202, which may be a microcatheter, for example, is positioned so that the distal tip 204 of the delivery catheter 202 is appropriately situated, for example within the mouth of the body cavity 201 to be treated. The insertion of the delivery catheter 202 may be facilitated by the use of a guidewire and/or a guiding catheter, as is known in the art. In addition, the movement of the catheter 202 may be monitored fluoroscopically.

Once the delivery catheter 202 is in place, the vaso-occlusive device 100 is inserted from the proximal end (not shown) of the delivery catheter 202 into the lumen of the delivery device 202. In one embodiment, since the vaso-occlusive device 100 has no secondary or tertiary relaxed configuration, the vaso-occlusive device 100 would naturally assume a substantially rectilinear or a curvilinear configuration when disposed within the lumen of the delivery device 202, without being subjected to substantial stress. In other embodiments, a secondary or tertiary “deployed” shape may be imposed, as is well-known.

In one embodiment, the vaso-occlusive device 100 is advanced distally towards the distal end 204 of the delivery catheter 202 with the use of a wire 206. A plunger 208 may be attached to the distal end of the wire 206 to assist advancement of the vaso-occlusive device 100. Alternatively, fluid pressure may also be used to advance the vaso-occlusive device 100 along the delivery catheter 202. The inner diameter of the delivery catheter 202 should be made large enough to allow advancement of the vaso-occlusive device 100. On the other hand, the inner diameter of the delivery catheter 202 should not be significantly larger than the overall cross-sectional dimension of the vaso-occlusive device 100 in order to avoid bending and kinking of the vaso-occlusive device 100 within the lumen of the delivery catheter 202.

For a vaso-occlusive device having no secondary or tertiary relaxed configuration, the vaso-occlusive device would remain substantially rectilinear or curvilinear without undergoing substantial stress, while residing within the lumen of the delivery catheter 202. Once the vaso-occlusive device 100 or a portion of the vaso-occlusive device 100 exits from the distal end 204 of the delivery catheter 202, it remains substantially rectilinear or curvilinear until it comes in contact with an object, i.e. the wall of the body cavity 201. If the vaso-occlusive device 100 is continued to be advanced towards the body cavity, the vaso-occlusive device 100 would be subjected to axial and/or bending stress due to the force/pressure exerted by the advancing force and by the object that it comes in contact with. As the result, the vaso-occlusive device 100 folds along one or more axes. When the vaso-occlusive device 100 is completely discharged from the delivery catheter 202, it assumes a three dimensional configuration within the body cavity 401. Optionally, the vaso-occlusive device 100 may be detached through operation of a mechanically or electrolytically detachable joint.

While certain exemplary embodiments have been described and shown in the accompanying drawings, it is to be understood that such embodiments are merely illustrative and not restrictive of the invention, and that various modifications may occur to those ordinarily skilled in the art upon studying this disclosure, without departing from the scope of the accompanying claims.

What is claimed is:

1. An embolic implant, comprising:
a tubular body having a plurality of slots formed therein, wherein respective connecting elements extend between adjacent segments of the tubular body, the respective slots and connecting elements configured to allow flexible bending of the implant, wherein adjacent connecting elements are rotated relative to each other to provide flexibility of the tubular structure along more than one axis of rotation.

2. The embolic implant of claim 1, the tubular body having a substantially circular cross-sectional shape.

3. The embolic implant of claim 1, further comprising one or more thrombogenic filamentary elements attached to the connecting elements of the tubular body.

4. The embolic implant of claim 1, wherein the tubular body comprises a biodegradable material.

5. The embolic implant of claim 1, wherein the tubular body comprises a polymer.

6. The embolic implant of claim 5, wherein the polymer is a shape memory polymer.

7. The embolic implant of claim 1, wherein the tubular body is impregnated and/or coated with an embolization agent.

8. The embolic implant of claim 1, wherein the tubular body is fluid swellable to expand at an in-situ location of the vasculature in which it is deployed.

9. The embolic implant of claim 1, wherein the tubular body has a lubricious coating to enhance deliverability.

10. The embolic implant of claim 1, wherein the tubular body comprises chemical sites capable of chelating radioisotopes for radioembolization.

11. An embolic implant, comprising:
a biodegradable tubular body having a substantially circular cross-sectional shape with a plurality of slots formed therein, wherein respective connecting elements extend between adjacent segments of the tubular body, the respective slots and connecting elements configured to allow flexible bending of the implant; and
one or more thrombogenic filamentary elements attached to the connecting elements of the tubular body.

12. The embolic implant of claim 11, wherein adjacent connecting elements are rotated relative to each other to provide flexibility of the tubular structure along more than one axis of rotation.

13. The embolic implant of claim 12, wherein the tubular body is impregnated and/or coated with an embolization agent.

14. The embolic implant of claim 13, wherein the tubular body is fluid swellable to expand at an in-situ location of the vasculature in which it is deployed.
